(19) United States
(12) Patent Application Publication
(10) Pub. No.: US 2008/0172099 A1
(43) Pub. Date: Jul. 17, 2008

(54) Medical Implant For Evoked Response Detection Having an Adaptive Detection Time Window

(76) Inventor: Anders Bjorling, Jarfalla (SE)

Correspondence Address: SCHIFF HARDIN, LLP;PATENT DEPARTMENT, 6600 SEARS TOWER, CHICAGO, IL, 60606-6473, US

(21) Appl. No.: 10/568,986

(22) PCT Filed: Aug. 28, 2003
(86) PCT No.: PCT/SE03/01340

§ 371 Date: Feb. 21, 2006

(51) Int. Cl. A61N 1/37

(52) U.S. Cl. 607/28

(57) ABSTRACT

A medical implant has a stimulation pulse generator and an evoked response detector that senses an IEGM signal in an evoked response detection time window following delivery of a stimulation pulse, in order to distinguish capture from loss of capture based on a parameter, from among a number of parameters, of the IEGM signal. A setting unit sets a minimum tolerable difference between the value of the selected parameter obtained as a result of capture and obtained as a result of loss of capture, respectively. The selected parameter that is used to distinguish capture from loss of capture can be the parameter for which the minimum tolerable difference is obtained with the shortest evoked response detection time window, or the parameter for which a calculated difference, between the value of the parameter resulting from capture and the value of the parameter resulting from loss of capture, has a maximum difference from the minimum tolerable difference.

### BACKGROUND OF THE INVENTION

1. Field of the Invention

The present invention relates to a medical implant of the type having a pulse generator for delivering stimulation pulses to at least one chamber of a patient's heart, an evoked response detector for distinguishing capture from loss of capture from the value of a selected one of a number of parameters obtainable from an IEGM signal sensed in an evoked response detection time window following delivery of a stimulation pulse, and a setting unit for setting a minimum tolerable difference between values of the selected parameter obtained in case of capture and in case of loss of capture, respectively. As an alternative, the evoked response detection window can have a fixed length.

2. Description of the Prior Art

Implantable pacemakers, which automatically detect capture and thereby minimize pacing energy, provide many benefits. The use of minimal pacing energy maximizes device longevity and minimizes the size of the device, and most importantly, automatic output regulation protects the patient from loss of capture caused by a rise in the threshold of stimulation.

For automatic capture a cardiac signal sensed in an evoked response detection time window after each stimulation pulse is analysed to determine whether or not the stimulation pulse captured the heart of a patient. The length of the evoked response detection time window is conventionally fixed. If a shorter evoked response detection time window could be used, a stimulation backup pulse could be delivered quicker, however, the shorter evoked response detection time window the greater risk of inaccurate decisions.

The shortest length of an evoked response detection time window that has a tolerable risk of inaccurate decisions depends on the lead type, the lead position, the evoked response of the patient and the parameter used to distinguish capture from loss of capture. Evoked response detection is the heart of the algorithm of automatic capture and thus very important.

There are mainly three different evoked response detection methods today, namely methods using the parameters maximum signal amplitude, maximum signal slope of the sensed IEGM signal, or area obtained by integrating the sensed IEGM signal over the evoked response detection time window. The value of the measured parameter is compared to a pre-set threshold. Values above the threshold indicate capture and values below the threshold indicate loss of capture. Thus, Boriani et. al. “Atrial Evoked Response Integral for Automatic Capture Verification in Atrial Pacing”, PACE 2003, Vol. 26, Part II, page 1-5, January 2003, describe the use of the integral of the atrial evoked response signal as a resource for verification of atrial capture.

An object of the present invention is to provide an improved medical implant which is quick in distinguishing capture from loss of capture and with a tolerable risk of inaccurate decisions.

The above object is achieved by a medical implant having a pulse generator for delivering stimulation pulses to at least one chamber of a patient's heart, an evoked response detector for distinguishing capture from loss of capture from the value of a selected one of a number of parameters obtainable from an IEGM signal sensed in an evoked response detection time window following delivery of a stimulation pulse, and a setting unit for setting a minimum tolerable difference between values of the selected parameter obtained in case of capture and in case of loss of capture, respectively, and having a first calculation unit that calculates for each of said parameters, the length of the evoked response detection time window for which the minimum tolerable difference is obtained, and a first selecting unit that selects that parameter for distinguishing capture and loss of capture for which the minimum tolerable difference is obtained with the shortest evoked response detection time window.

Thus, this medical implant is able to automatically select that parameter for distinguishing capture and loss of capture for which the minimum tolerable difference is obtained with the shortest evoked response detection time window. The only requirements are that the evoked response detector is able to distinguish capture from loss of capture with at least one of the available parameters if an evoked response detection time window of a maximum length is used, where the maximum length can be very large, e.g. 120 ms, and that a minimum tolerable difference between values of the selected parameter obtained in case of capture and in case of loss of capture, respectively, is set.

In an embodiment of the medical implant according to the present invention, a third calculation unit is provided to calculate a matrix or table of the difference for different lengths of the evoked response detection time window and different ones of said parameters for storage for use in later off-line analysis.

The above object also is achieved by a medical implant according to the invention, having a second calculation unit that calculates, for each of the parameters, the difference between the value of the parameter obtained in case of capture and in case of loss of capture, respectively, and a second selecting unit that selects that parameter for distinguishing capture and loss of capture by comparison with the minimum tolerable difference for which a maximum difference is obtained. In this way the risk of inaccurate decision is reduced to a minimum.

In an embodiment of the medical implant according to the invention, said setting unit sets the minimum tolerable difference with a safety margin. In a further embodiment of the medical implant according to the invention, the minimum tolerable difference is pre-set or programmable.

In another embodiment of the medical implant according to the invention, the setting unit and the second calculation unit calculate, as the aforementioned difference the signal-to-noise-ratio SNR from the equation

$$SNR = \{ \begin{matrix}\frac{min ( {ERM}_{capture} ) - max ( {ERM}_{lossofcapture} )}{max ( {ERM}_{capture} ) - min ( {ERM}_{lossofcapture} )} , & {\overline{ERM}}_{capture} > {\overline{ERM}}_{lossofcapture} \\ \frac{max ( {ERM}_{capture} ) - min ( {ERM}_{lossofcapture} )}{min ( {ERM}_{capture} ) - max ( {ERM}_{lossofcapture} )} , & {\overline{ERM}}_{capture} < {\overline{ERM}}_{lossofcapture}\end{matrix} \} \\ {\overline{ERM}}_{capture} ≡ \frac{1}{N} {\sum }_{i = 1}^{N} {ERM}_{capture} ( i ) \qquad Equation [ 1 ]$$

where ERM_{capture} and ERM_{loss of capture} or capture denote the parameter values obtained in case of capture and loss of capture, respectively.

In another embodiment of the medical implant according to the invention, the parameters include maximum signal amplitude and maximum signal slope of the sensed IEGM signal, and area obtained by integrating the sensed IEGM signal over the evoked response detection time window.

In a further embodiment of the medical implant according to the invention, a differentiating unit is provided to calculate the derivative of the sensed IEGM signal for the determination of the maximum slope.

In a further a further embodiment of the medical implant according to the invention, the pulse generator is controlled to deliver a stimulation back-up pulse at the end of the evoked response detection time window in response to detected loss of capture.

### DESCRIPTION OF THE DRAWINGS

FIG. 1 is a diagram showing a sensed evoked response signal resulting from a stimulation pulse.

FIG. 2 shows schematically a first preferred embodiment of the medical implant according to the present invention.

FIG. 3 shows schematically a second preferred embodiment of the medical implant according to the present invent.

FIG. 4 shows a flow diagram of a procedure performed by the first preferred embodiment of the medical implant according to the present invention.

FIG. 5 shows a flow diagram of a procedure performed by the second preferred embodiment of the medical implant according to the present invention.

### DESCRIPTION OF THE PREFERRED EMBODIMENTS

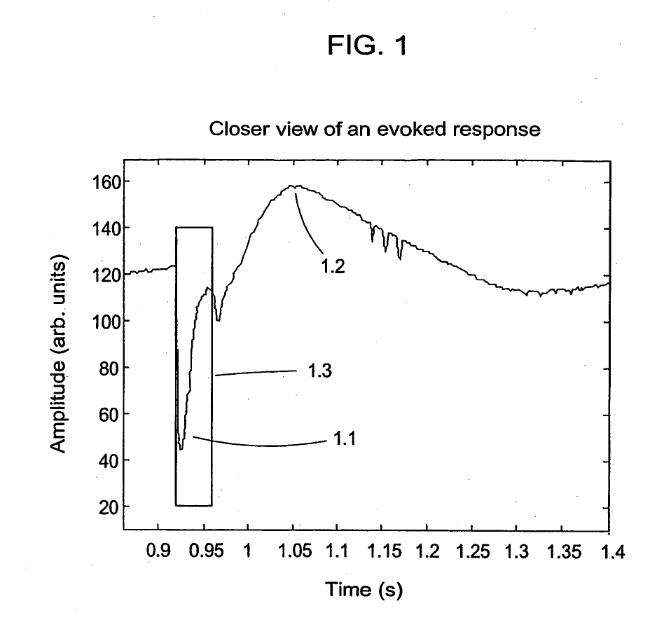

FIG. 1 shows an evoked response resulting from a stimulation pulse. In the diagram an evoked response 1.1 is shown, followed by a T wave 1.2. Further, an evoked response detection time window 1.3 is shown as a rectangle. The signal sensed in this time window 1.3 is analysed to determine whether or not the stimulation pulse has captured the heart. Herein, the length of the window 1.3 is about 39 ms.

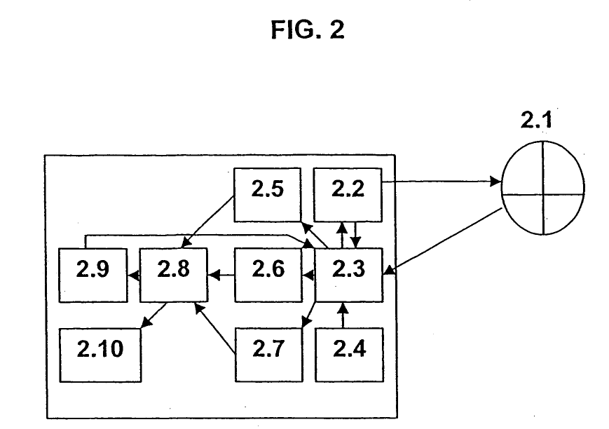

FIG. 2 illustrates schematically a first preferred embodiment of the medical implant according to the present invention. The medical implant is connected to a patient's heart 2.1 and has a pulse generator 2.2 for delivering stimulation pulses to at least one chamber of the patient's heart 2.1. The pulse generator 2.2 is controlled to deliver a stimulation back-up pulse at the end of the evoked response detection time window in response to detected loss of capture. The medical implant also comprises an evoked response detector 2.3 for distinguishing capture from loss of capture from the value of a selected one of a plurality of parameters obtainable from an IEGM signal sensed in an evoked response detection time window, as shown in FIG. 1, following delivery of a stimulation pulse. These parameters include maximum signal amplitude and maximum signal slope of the sensed IEGM signal, and area obtained by integrating the sensed IEGM signal over the evoked response detection time window. Further, the medical implant comprises a setting unit 2.4 for setting a minimum tolerable difference between values of the selected parameter obtained in case of capture and in case of loss of capture respectively. The setting unit 2.4 sets the minimum tolerable difference with a safety margin. The minimum tolerable difference is pre-set or programmable. The setting unit 2.4 calculates, as the difference, the signal-to-noise-ratio SNR from above-mentioned Equation [1]. A differentiating unit 2.5 calculates the derivative of the sensed IEGM signal for the determination of the maximum slope, an integrating unit 2.6 integrates the sensed IEGM signal over the evoked response detection window providing above-said area, and a maximum signal amplitude unit 2.7 provides the maximum signal amplitude. A first calculation unit 2.8 calculates, for each of the parameters the length of the evoked response detection time window for which the minimum tolerable difference is obtained, together with a first selecting unit 2.9 that selects that parameter for distinguishing capture and loss of capture for which the minimum tolerable difference is obtained with the shortest evoked response detection time window. Further, a third calculation unit 2.10 calculates a matrix or table of the difference for different lengths of the evoked response detection time window and different ones of the parameters for storage for use in later off-line analysis.

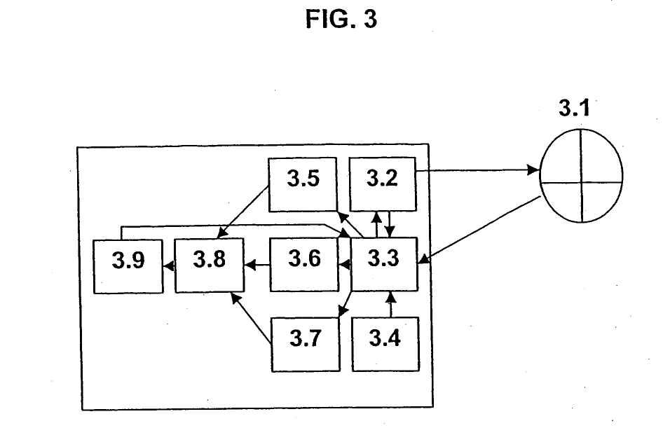

FIG. 3 illustrates schematically a second preferred embodiment of the medical implant according to the present invention. As the embodiment of FIG. 2, this embodiment also has a pulse generator 3.2, an evoked response detector 3.3, a setting unit 3.4, a differentiating unit 3.5, an integrating unit 3.6, and a maximum signal amplitude unit 3.7, each with the same function as in the embodiment of FIG. 2. In this embodiment the evoked response detection time window has a fixed length. A second calculation unit 3.8 calculates, for each of the parameters, the difference between the value of the parameter obtained in case of capture and in case of loss of capture, respectively, and the second calculation unit 3.8 calculates, as the difference, the signal-to-noise-ratio SNR from above-mentioned Equation [1]. Further, a second selecting unit 3.9 selects that parameter for distinguishing capture and loss of capture by comparison with the minimum tolerable difference for which a maximum difference is obtained.

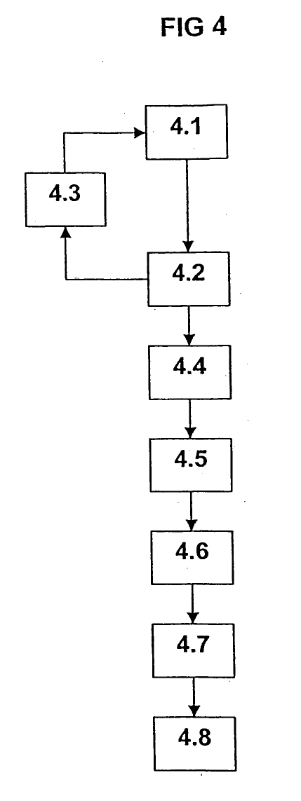

FIG. 4 is a flow diagram illustrating a procedure performed by the above-mentioned first preferred embodiment of the medical implant according to the present invention, where the procedure includes the following steps:

4.1 Delivering a series of stimulation pulses to at least one chamber of a patient's heart, the amplitude of which ranging from zero to a certain maximum amplitude.

4.2 Recording the electrical activity in an evoked response time window of a certain maximum length after each stimulation pulse. The recording is performed as a modified VARIO test from the maximum amplitude down to zero without interrupting it.

4.3 Emitting a backup pulse at the end of the evoked response time window after each stimulation pulse.

4.4 Storing the electrical activity in an evoked response time window of a certain maximum length.

4.5 After completion of the recording, calculating the parameters for the evoked response time window of said certain maximum length from the stored values.

4.6 Determining the stimulation threshold for capture.

4.7 Calculating the signal-to-noise-ratio SNR from the above-mentioned Equation [1] for all parameters and multiple evoked response time window lengths.

4.8 Selecting that parameter for distinguishing capture from loss of capture for which the SNR is above a pre-set minimum tolerable difference with the shortest evoked response detection time window.

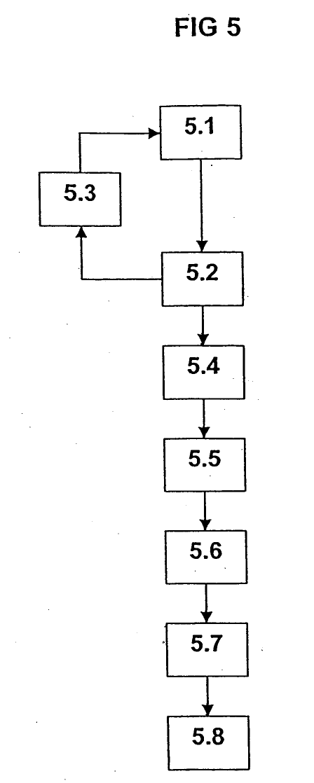

FIG. 5 is a flow diagram illustrating a procedure performed by the second preferred embodiment of the medical implant according to the present invention. The length of the evoked response detection window is specified and fixed, and the procedure includes the steps 5.1 to 5.7, which correspond to the steps 4.1 to 4.7 of the procedure of FIG. 4, and step 5.8, which involves selecting that parameter for distinguishing capture and loss of capture by comparison with said minimum tolerable difference for which the greatest SNR is obtained is done.

Although modifications and changes may be suggested by those skilled in the art, it is the intention of the inventors to embody within the patent warranted hereon all changes and modifications as reasonably and properly come within the scope of their contribution to the art.

1-10. (canceled)

11. A medical implant comprising:
a pulse generator adapted to interact with at least one chamber of a heart to deliver stimulation pulses to said at least one chamber;
an evoked response detector adapted to interact with the heart to sense an IEGM signal therefrom in an evoked response detection time window following delivery of a stimulation pulse by said pulse generator, said sensed IEGM signal embodying a plurality of parameters and said evoked response detector distinguishing capture from loss of capture from a value of a selected one of said plurality of parameters;
a setting unit that sets, for said selected one of said plurality of parameters, a minimum tolerable difference between a value of the selected parameter obtained as a result of capture and a value of said selected parameter obtained as a result of loss of capture;
a calculation unit that calculates, for each parameter in said plurality of parameters, a length of the evoked response detection time window for which said minimum tolerable difference is obtained; and
a selecting unit that selects said one of said plurality of parameters, as the parameter for which said minimum tolerable difference is obtained with the shortest evoked response detection time window, as calculated by said calculation unit.\

12. A medical implant as claimed in claim 11 wherein said setting units sets said minimum tolerable difference with a safety margin.

13. A medical implant as claimed in claim 11 comprising a further calculation unit that compiles a compilation of said minimum tolerable difference for different lengths of said evoked response detection time window and different ones of said plurality of parameters, and stores said compilation in a memory accessible for subsequent off-line analysis.

14. A medical implant as claimed in claim 11 wherein said minimum tolerable difference is pre-set is said setting unit.

15. A medical implant as claimed in claim 11 wherein said minimum tolerable difference is programmable in said setting unit.

16. A medical implant as claimed in claim 11 wherein said parameters comprise a maximum signal amplitude of the sensed IEGM signal, a maximum signal slope of the sensed IEGM signal, and an area obtained by integrating the sensed IEGM signal over said evoked response detection time window.

17. A medical implant as claimed in claim 16 comprising a differentiating unit supplied with said sensed IEGM signal that calculates the first derivative with respect to time of said sensed IEGM signal, as said maximum signal slope.

18. A medical implant as claimed in claim 11 comprising a control unit connected to said pulse generator and to said evoked response detector, said control unit controlling said pulse generator to cause said pulse generator to deliver a stimulation back-up pulse at an end of said evoked response detection time window if loss of capture is detected by said evoked response detector.

19. A medical implant comprising:
a pulse generator adapted to interact with at least one chamber of a heart to deliver stimulation pulses to said at least one chamber;
an evoked response detector adapted to interact with the heart to sense an IEGM signal therefrom in an evoked response detection time window following delivery of a stimulation pulse by said pulse generator, said sensed IEGM signal embodying a plurality of parameters and said evoked response detector distinguishing capture from loss of capture from a value of a selected one of said plurality of parameters;
a setting unit that sets, for said selected one of said plurality of parameters, a minimum tolerable difference between a value of the selected parameter obtained as a result of capture and a value of said selected parameter obtained as a result of loss of capture;
a calculation unit that calculates, for each parameter in said plurality of parameters, a calculated difference between a value of the parameter obtained as a result of capture and a value of the parameter obtained as a result of loss of capture; and
a selecting unit that compares, for each parameter in said plurality of parameters, the calculated difference with said minimum tolerable difference and that selects said one of said plurality of parameters as the parameter for which a maximum difference exists between the calculated difference and the minimum tolerable difference.

20. A medical implant as claimed in claim 19 wherein said setting units sets said minimum tolerable difference with a safety margin.

21. A medical implant as claimed in claim 19 wherein said minimum tolerable difference is pre-set is said setting unit.

22. A medical implant as claimed in claim 19 wherein said minimum tolerable difference is programmable in said setting unit.

23. A medical implant as claimed in claim 19 wherein said setting unit and said calculation unit calculates said calculated difference as a signal-to-noise ratio SNR, according to
$$SNR = \{ \begin{matrix}\frac{min ( {ERM}_{capture} ) - max ( {ERM}_{lossofcapture} )}{max ( {ERM}_{capture} ) - min ( {ERM}_{lossofcapture} )} , & {\overline{ERM}}_{capture} > {\overline{ERM}}_{lossofcapture} \\ \frac{max ( {ERM}_{capture} ) - min ( {ERM}_{lossofcapture} )}{min ( {ERM}_{capture} ) - max ( {ERM}_{lossofcapture} )} , & {\overline{ERM}}_{capture} < {\overline{ERM}}_{lossofcapture}\end{matrix} \}$$
$${\overline{ERM}}_{capture} ≡ \frac{1}{N} {\sum }_{i = 1}^{N} {ERM}_{capture} ( i )$$
wherein ERM_{capture} is the value of the parameter obtained as a result of capture and ERM_{loss of capture} is the value of the parameter obtained as a result of loss of capture.

24. A medical implant as claimed in claim 19 wherein said parameters comprise a maximum signal amplitude of the sensed IEGM signal, a maximum signal slope of the sensed IEGM signal, and an area obtained by integrating the sensed IEGM signal over said evoked response detection time window.

25. A medical implant as claimed in claim 24 comprising a differentiating unit supplied with said sensed IEGM signal that calculates the first derivative with respect to time of said sensed IEGM signal, as said maximum signal slope.

26. A medical implant as claimed in claim 19 comprising a control unit connected to said pulse generator and to said evoked response detector, said control unit controlling said pulse generator to cause said pulse generator to deliver a stimulation back-up pulse at an end of said evoked response detection time window if loss of capture is detected by said evoked response detector.
